(19) United States
(12) Patent Application Publication
(10) Pub. No.: US 2022/0265981 A1
(43) Pub. Date: Aug. 25, 2022

(54) Body-Worn Therapeutic Device

(71) Applicant: Kellie Glover, Shepherdsville, KY (US)

(72) Inventor: Kellie Glover, Shepherdsville, KY (US)

(21) Appl. No.: 17/677,002

(22) Filed: Feb. 22, 2022

Related U.S. Application Data
(60) Provisional application No. 63/152,552, filed on Feb. 23, 2021.

(51) Int. Cl. A61M 35/00; A61F 7/02; A61H 99/00

(52) U.S. Cl. CPC A61M 35/10; A61F 7/02; A61H 99/00; A61F 2007/0238; A61H 2201/165; A61F 2007/0094

(57) ABSTRACT

The present invention relates generally to the field of therapeutic accessories. More specifically, the present invention relates to a body-worn accessory which can be used to provide heating or massaging to an area of the body. The invention has a body with a front surface, a rear surface and an interior surface, a battery and a remote. The interior surface of the body has at least one heating or massaging element that is powered by the battery, and can be controlled by the remote. The remote further allows a user to control the heat or massage level of the at least one pad and the activity/inactivity of the element. The body of the device may also have a pouch to store one of a battery, pad or sponge.

### CROSS-REFERENCE TO RELATED APPLICATION

The present application claims priority to, and the benefit of, U.S. Provisional Application No. 63/152,552, which was filed on Feb. 23, 2021, and is incorporated herein by reference in its entirety.

### FIELD OF THE INVENTION

The present invention relates generally to the field of treatment accessories for individual comfort. More specifically, the present invention relates to a body-worn heating and/or massaging accessory. The invention has a body that has a front surface, a rear surface and an interior surface, a battery and a remote control to operate the features of the heating or massaging accessory. The interior surface of the body includes at least one heating or massaging element that is powered by the battery and can be operated by the remote control. The remote control further allows a user to change the heat or vibration level of the at least one heating or massaging element, and provides a timer in order to calculate the activity or inactivity of the heating or massaging accessory. Further, the body of the invention may have a pouch to store the battery. The invention can be comfortably and discretely worn by a user to provide all-day heat comfort and relief to the front torso, rear torso and perineum of a user, and is portable. Accordingly, the present disclosure makes specific reference thereto. Nonetheless, it is to be appreciated that aspects of the present invention are also equally applicable to other like applications, devices and methods of manufacture.

### BACKGROUND

Many individuals suffer from medical conditions or ailments that affect the torso area of their bodies. Such painful conditions or ailments may include menstrual cramps, kidney pain, bladder pain, ovarian cysts, endometriosis, back pain, other aches, chills caused by poor circulation, arthritic and other conditions. These conditions may further respond well to heat therapy, which may soothe and provide pain relief to a user. In order to apply heat to the torso area, a user may apply one or more heating packs. However, these packs must be manually held or secured such as by tape or wraps to the desired body area of the user. As a result, an individual cannot easily wear the heating packs throughout a day as the packs may easily become unsecured, are bulky and simply not convenient to use in an active lifestyle. Further, the packs may be visible under the clothing of the user, which is also undesirable. In addition, existing heating packs known in the art often only have one heating level, which cannot be adjusted by a user. Both outcomes are undesirable, as some pain is so severe that a user cannot perform daily tasks and functions without heat pack relief, and existing heat packs may also be too hot for the preference of the user. In addition, when attempt to apply a massaging treatment to the body, the massagers are typically hand-held and difficult to carry and use when needed.

Therefore, there exists a long-felt need in the art for an improved heating and therapeutic device. There also exists a long-felt need in the art for a body-worn heating and massaging pad and therapeutic device that can be discretely, comfortably and securely worn on the body of a user throughout the day. Further, there exists a long-felt need in the art for a body-worn heating, massaging and therapeutic device that offers a plurality of heat and massaging levels to be selected by a user.

The subject matter disclosed and claimed herein, in one embodiment thereof, comprises a body-worn heating pad and therapeutic device. The invention has a body with a front surface, a rear surface and an interior surface, a battery and a remote control. The interior surface of the body portion has at least one heating or massaging element that is powered by the battery and can be controlled or operated by the remote. The remote control further allows a user to modify the heat or massaging level of the at least one heating element and a timer to control the activity or inactivity of the invention. Further, the body of the device may have a pouch to store the battery. The device can be comfortably and discretely worn by a user to provide all-day heat or massage relief to the front torso, rear torso and perineum of a user. The body may also contain elastic areas, reinforced areas that can be used to provide additional comfort and therapeutic comfort to the individual, as well as removable pads for lotions, creams or essential oils.

In this manner, the body-worn heating device of the present invention accomplishes all of the forgoing objectives and provides an improved heating accessory that can be worn on the body of a user. The device can be discretely and securely worn on the body of a user throughout the day, such that it can continuously or selectively apply heat or massage to areas of the body of the user as desired. In addition, the device offers a plurality of heat or massaging levels that can be selected by a user for increased comfort and customization. The therapy device can also provide vibration panels to help in soothing pains or aches of the individual. Therefore, the device overcomes the limitations of existing heating pads known in the art.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some general concepts in a simplified form as a prelude to the more detailed description that is presented later.

The subject matter disclosed and claimed herein, in one embodiment thereof, comprises a body-worn heating and/or massaging accessory having a body that has a front surface, a rear surface and an interior surface, a battery and a remote. The body is manufactured from a durable fabric, and in one embodiment the interior surface is preferably manufactured from a cotton or microfiber fabric. In addition, the body may be sweatproof, waterproof and have antibacterial and or antimicrobial properties. The body has two continuous leg or hip openings that allow the legs of a user to slip through the invention when the device is worn by a user. To fasten the invention to a user, the rear surface has at least two fastening tabs that may be fixedly or removably-attached to the rear surface and can then be removably secured to the front surface. The tabs may have numerous methods of attachment such as, but not limited to, a hook and loop, adhesive, magnet, snap button, zipper, ties or combinations thereof. The body also includes one or more pouches that allow a battery to be stored on the body as well as other treatment pads or sponges to hold oils, creams, lotions or combinations thereof. The pouches may have at least one fastener that may be any fastener known in the art such as, but not limited to, a hook and loop, adhesive, magnet, snap button, zipper, ties, etc., to secure the battery, pads or sponges within the pouches. In differing embodiments, the battery may be disposable or rechargeable via a USB charging port. The pads or sponges may be reusable, washable and otherwise interchangeable.

The battery further supplies power to and is in electrical communication with at least one heating element and/or vibration pad that is located within the interior surface and adjacent to the body of the individual. The invention preferably has three or more heating pads and/or vibration-providing areas that apply heat or vibration to the front torso, rear torso and perineum area of a wearer. Using a remote control, a user can select which of the pads is active, and at what heat level the pads output heat (ex. heat level 1, heat level 2, heat level 3, etc. and/or low, medium, high or in case of the vibration, high, medium and low). The remote control preferably has at least one button that allows a user to: select which heating or vibration pad is active, select which heating or vibration pad is not active, select a heat or vibration intensity level for each pad and set a timer for how long the pads remains active. Therefore, a user can configure the pads to provide a plurality of different heat or vibration levels to various portions of the torso and body as needed throughout a day.

Accordingly, the body-worn therapeutic device of the present invention is particularly advantageous as it provides an improved treating device which can provide both heat and vibration to address any aliment of an individual. The device can be discretely and securely worn on the body of a user throughout the day, such that it can continuously or selectively apply heat or vibrations to the body of the user. Further, the device offers a plurality of heat or vibration levels that can be selected by a user for increased comfort and customization. In this manner, the device overcomes the limitations of existing heating pads, packs and massagers known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a body-worn therapeutic device of the present invention in an unworn and unfastened state in accordance with the disclosed architecture;

FIG. 2 illustrates a perspective view of one potential embodiment of a body-worn therapeutic device of the present invention in an unworn and fastened state in accordance with the disclosed architecture; and

FIG. 3 illustrates a perspective view of one potential embodiment of a body-worn therapeutic device of the present invention being worn by a user in accordance with the disclosed architecture.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an improved therapeutic device. There also exists a long-felt need in the art for a body-worn therapeutic device that can be discretely and securely worn on the body of a user throughout the day. Further, there exists a long-felt need in the art for a body-worn therapeutic device that offers a plurality of heat and massaging levels that can be selected by a user, as well as other treatment pads for holding creams, oils, lotions, ointments or combinations thereof.

The present invention, in one exemplary embodiment, is comprised of a body-worn therapeutic device which has a body that has a front surface, a rear surface and an interior surface, a battery and a remote. The body is manufactured from a durable fabric, and in one embodiment the interior surface is preferably made from a cotton or microfiber fabric. The body may also be sweatproof, waterproof and have antibacterial and or antimicrobial properties. In addition, the body has two continuous leg or hip openings that allow the legs of a user to slip through the body when the device is worn by a user. To fasten the device to a user, the rear surface has at least two fastening tabs that may be fixedly or removably-attached to the rear surface, and can then be removably-secured to the front surface. The tabs may have numerous methods of attachment such as, but not limited to, a hook and loop, adhesive, magnet, snap button, zipper, ties or combinations thereof. The front or interior surface of the body has one or more pouches that allows a battery to be stored on the body as well as other sponges, pads or the like to hold oils, lotions, creams, ointments and combinations thereof, to aid in treating various aliments of the individual. The pouches may have at least one fastener that may be any fastener known in the art such as, but not limited to, a hook and loop, adhesive, magnet, snap button, zipper, ties or combinations thereof to secure the battery or other sponges, pads or other items within the pouches. In differing embodiments, the battery may be disposable or rechargeable via a USB charging port. The pads or sponges may be washable, disposable and are intended to be interchangeable.

The battery further supplies power to and is in electrical communication with at least one heating element or massaging element that is located within the interior surface of the body. The device preferably has three or more heating or massaging pads that apply heat or vibrations to the front torso, rear torso and perineum area of a wearer. Using a remote control, a user can select which of the pads is active, and at what heat or massaging level the pads output heat (ex. heat level 1, heat level 2, heat level 3, etc., and/or low, medium, high, vibration high, medium, low). The remote control preferably has at least one button that allows a user to: select which heating or massaging pad is active, select which heating or massaging pad is not active, select a heat or massage intensity level for the pads and set a timer for how long the pads remain active. Therefore, a user can configure the pads to provide a plurality of different heat or massaging levels to various portions of the torso and body as needed throughout a day, and the user can selectively activate some pads to provide heat and others to provide a massage feature.

Accordingly, the body-worn therapeutic device of the present invention is particularly advantageous as it provides an improved treatment device that can be discretely and securely worn on the body of a user throughout the day, such that it can continuously or selectively apply heat or massage to the body of the user. Further, the device offers a plurality of heat or vibration levels that can be selected by a user for increased custom ability. In this manner, the device overcomes the limitations of existing heating pads/packs and massage units known in the art.

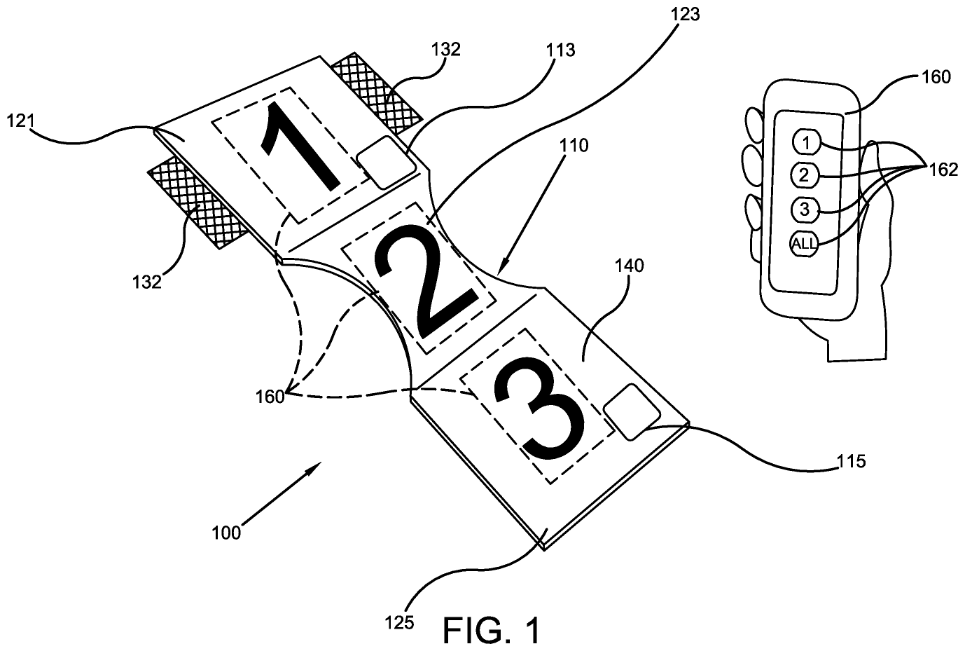

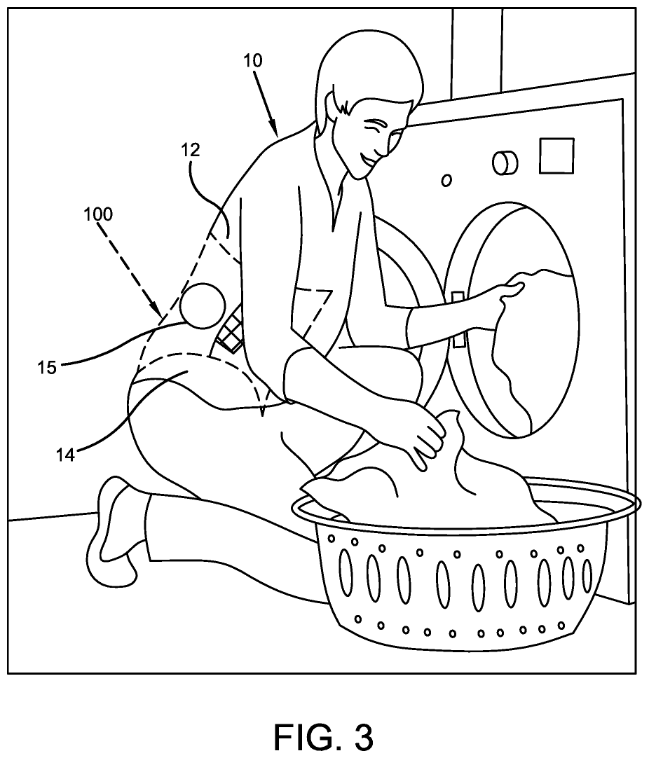

Referring initially to the drawings, FIG. 1 illustrates a perspective view of one potential embodiment of a body-worn therapeutic device 100 of the present invention in an unworn and unfastened state in accordance with the disclosed specification. The invention 100 has a body 110 with a front surface 120, a rear surface 130 and an interior surface 140, a battery 150 and a remote 160. In differing embodiments, the body 110 may have a plurality of fabrics such as, but not limited to, a vinyl, canvas, cashmere, chenille, chiffon, cotton, damask, jersey, lace, linen, wool, modal, polyester, satin, silk, spandex, suede, tweed, twill, velvet, acrylic, modacrylic, nylon, polypropylene, polyurethane, polyvinyl chloride, polyethylene, vinylidene, benzoate, aramid, rayon, acetate, triacetate, etc. In addition, the interior surface 140 is preferably manufactured from a comfortable material such as, but not limited to, cotton or microfiber. In any embodiment, the body 110 may also be sweatproof, waterproof and have antibacterial and/or antimicrobial properties. When worn by a user 10, the body 110 has two continuous leg/hip openings 112 that allow the legs of a user to slip through the body 110 (as seen in FIG. 3). The interior surface of the invention may also be provided with pouches 113 and 115 which may be positioned anywhere on the surface. The pouches 113, 115 may hold sponges, pads or the like which can be used to hold lotions, creams, ointments, essential oils or combinations thereof. Essential oils such as rose, ginger, peppermint, eucalyptus, lavender, sandalwood, myrrh, thyme, wintergreen and lemongrass may be used in the various patches, sponges or pads. The body 110 has first, second and third panels 121, 123 and 125, each of which may contain one or more pads 142 providing heating or massaging therapeutic treatment to the individual user.

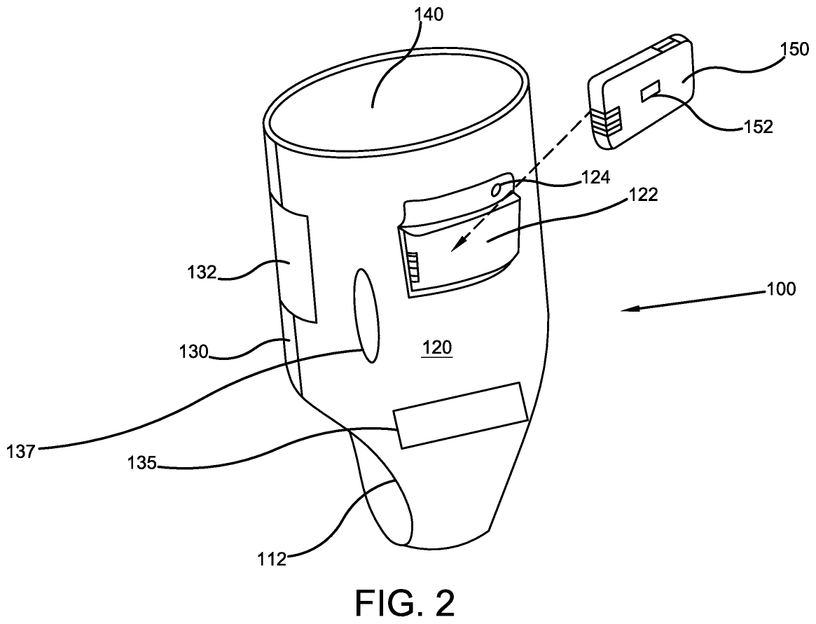

FIG. 2 illustrates a perspective view of one potential embodiment of a body-worn therapeutic device 100 of the present invention in an unworn and fastened state in accordance with the disclosed specification. To fasten the device 100 such that it can be securely worn by an individual 10, the rear surface 130 of the body 110 has at least two fastening tabs 132. The tabs 132 may be fixedly or removably-attached to the rear surface 130, and can then be removably-secured to the front surface 120. In differing embodiments, the tabs 132 may have numerous methods of attachment such as, but not limited to, a hook and loop, adhesive, magnet, snap button, zipper, ties, and combinations thereof. The front surface 120 has a pouch 122 that allows a battery 150 or other materials to be stored on the body 110. In order to secure the battery 150 within the pouch 122, the pouch may have at least one fastener 124 that may be any fastener known in the art such as but not limited to: hook and loop, adhesive, magnet, snap button, zipper, ties, etc. The body 110 may also have tension areas 135, 137 which can contain elastic, reinforced areas or the like which can be used to apply pressure to the select areas of the body.

The battery 150 may be a disposable battery or a rechargeable battery in the form of an alkaline, nickel-cadmium, nickel-metal hydride battery, etc., such as any 3V-12volts DC battery or other conventional battery such as A, AA, AAA, etc. that supplies power to the device 100. Throughout this specification the term “battery” may be used interchangeably to refer to one or more wet or dry cells or batteries of cells in which chemical energy is converted into electricity and used as a source of DC power. References to recharging or replacing the battery 150 may refer to recharging or replacing individual cells, individual batteries of cells, or a package of multiple battery cells as is appropriate for any given battery technology that may be used. The battery 150 may further contain a USB port 152 that allows the battery 150 to be recharged via a USB connection such as, but not limited to, a USB-A, USB-B, Micro-B, Micro-USB, Mini-USB, USB-C or a 9v/12v power supply.

The battery 150 further supplies power to and is in electrical communication with at least one pad 142 which can be used to provide heating or massaging that is located within the interior surface 140. In the preferred embodiment, the device 100 is comprised of three or more heating or massaging pads 142 that apply heat or vibrations to the front torso, rear torso, a perineum and/or a groin area of the individual 10. Using a remote 160, a user can select which of the pads 142 is active, and at what heat or massaging level the pads 142 output heat (ex. heat level 1, heat level 2, heat level 3, etc. and/or low, medium, high) or massage (high, medium or low). The remote 160 has at least one button 162, which allows a user to select which pad 142 is active, select which pad 142 is not active, select a heat or massage intensity level for the each individual or all pads 142 and set a timer for how long the pad(s) 142 remains active. In this manner, a user 10 can configure the pad or pads 142 to provide a plurality of different levels of therapeutic treatment to various portions of the torso 12 and body as needed throughout a day.

FIG. 3 illustrates a perspective view of one potential embodiment of a body-worn therapeutic device of the present invention while being worn by a user in accordance with the disclosed specification. As noted, the device 100 can be easily fastened around the torso 12 of an individual 10 such that the device 100 can be discretely worn under the clothes of a user. In this manner, a user 10 can enjoy all-day relief from the heating and massaging pads 142. The invention 100 can have various other treatment zones 15 which can be used to apply tension, bracing or reinforcement to areas of the garment.

The invention 100 is highly portable, and may be worn under the clothes. Additionally, battery 150 should be relatively slim and may be placed in a small battery pack pocket that can be removed for machine or hand washing. The battery 150 may be easily recharged and fastened so that the device 100 can be worn on the go, around the house, etc.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “body-worn heating pad device” and “device” are interchangeable and refer to the body-worn heating pad device 100 of the present invention.

Notwithstanding the forgoing, the body-worn heating pad device 100 of the present invention and its various components can be of any suitable size and configuration as is known in the art without affecting the overall concept of the invention, provided that they accomplish the above-stated objectives. One of ordinary skill in the art will appreciate that the size, configuration and material of the body-worn heating pad device 100 as shown in the FIGS. are for illustrative purposes only, and that many other sizes and shapes of the body-worn heating pad device 100 are well within the scope of the present disclosure. Although the dimensions of the body-worn heating pad device 100 are important design parameters for user convenience, the body-worn heating pad device 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A therapeutic device comprising:
a fabric body having a front surface, a rear surface and a fastening element provided on one of the front or rear surfaces to fasten the fabric body around a wearer;
at least one pouch provided on one of the front and rear surfaces of the fabric body;
at least one therapeutic pad disposed on an interior area of the fabric body; and
a remote control for operating the at least one therapeutic pad.

2. The therapeutic device as recited in claim 1, wherein the fabric body is comprised of a first panel, a second panel and a third panel.

3. The therapeutic device as recited in claim 2, wherein each of the first, second and third panels have a therapeutic pad.

4. The therapeutic device as recited in claim 3, wherein each of the first, second and third panels have one of a heating pad or a massage pad.

5. The therapeutic device as recited in claim 4, wherein the remote control can operate one or more of the first, second and third panels.

6. The therapeutic device as recited in claim 5, wherein the remote control can variably select a heat level or a massage level.

7. The therapeutic device as recited in claim 2, wherein at least one of the first, second and third panels comprise a pouch for holding at least one of a sponge or pad.

8. The therapeutic device as recited in claim 7, wherein the at least one of the sponge or pad contains at least one of an essential oil, an ointment, a lotion, a creams and a combination thereof.

9. The therapeutic device as recited in claim 8, wherein the essential oil is comprised of at least one of a rose, a ginger, a peppermint, an eucalyptus, a lavender, a sandalwood, a myrrh, a thyme, a wintergreen and a lemongrass.

10. The therapeutic device as recited in claim 1, wherein the fabric body comprises at least one of a sweatproof property, a waterproof property, an antibacterial property and an antimicrobial property.

11. A body warming device comprising:
a cover having a front surface and a back surface, wherein the front surface comprises a first panel, a second panel and a third panel;
a fastening element for securing the front surface and the back surface together around a torso of an individual, wherein at least one of the first, second and third panels comprise a controllable element; and
a remote control for controlling the controllable element.

12. The body warming device as recited in claim 11, wherein the controllable element is a heating element, a massaging element or a combination thereof.

13. The body warming device as recited in claim 12, wherein the controllable element is a heating element having a high setting, a medium setting and a low setting.

14. The body warming device as recited in claim 12, wherein the controllable element comprises an activity setting.

15. The body warming device as recited in claim 14, wherein the activity setting includes a timer to set a time to allow the controllable element to operate.

16. The body warming device as recited in claim 11, wherein the cover is made from one of a vinyl, a canvas, a cashmere, a chenille, a chiffon, a cotton, a damask, a jersey, a lace, a linen, a wool, a modal, a polyester, a satin, a silk, a spandex, a suede, a tweed, a twill, a velvet, an acrylic, a modacrylic, a nylon, a polypropylene, a polyurethane, a polyvinyl chloride, a polyethylene, a vinylidene, a benzoate, an aramid, a rayon, an acetate or a triacetate material.

17. The body warming device as recited in claim 11, wherein the fastening element is one of a hook and loop, an adhesive, a magnet, a snap button, a zipper or a tie fastening means.

18. The body warming device as recited in claim 11, wherein the cover further comprises at least one pouch for storing at least one of a battery, a sponge or a pad.

19. The body warming device as recited in claim 18, wherein the at least one of a sponge or a pad holds at least one of an essential oil, an ointment, a lotion, a cream and a combination thereof.

20. A body therapeutic device for surrounding a human torso, the body therapeutic device comprising:
a cover formed from a soft material and having a front surface and a rear surface, wherein the front and rear surfaces are joined to one another by a fastening element;
the cover having a first panel, a second panel and a third panel, each comprising a controllable element for providing a heating feature, a massaging feature or a combination thereof; and
a remote control for determining a setting for each of the controllable elements, wherein the setting comprises an activity setting for determining a length of a therapeutic treatment.
